(19) United States
(12) Patent Application Publication
(10) Pub. No.: US 2025/0018128 A1
(43) Pub. Date: Jan. 16, 2025

(54) Medicament delivery device with permanent locking of release button

(71) Applicant: SHL Medical AG, Zug (CH)

(72) Inventors: Daniel Carlsson, Nacka Strand (SE); William Alejandro Serna Campuzano, Medelin (CO)

(21) Appl. No.: 18/706,917

(22) PCT Filed: Oct. 21, 2022
(86) PCT No.: PCT/EP2022/079401

§ 371 Date: May 2, 2024

(30) Foreign Application Priority Data
Nov. 3, 2021 (EP) 21206214.5

(51) Int. Cl. A61M 5/315

(52) U.S. Cl. CPC A61M 5/3158; A61M 2205/583

(57) ABSTRACT

The present disclosure provides a power pack for a medicament delivery device. The power pack includes a tubular body with a first protrusion and a first contact surface, and a release button with a flexible arm including a second protrusion with a second contact surface and a third protrusion with a third contact surface. In a first position, the first and second contact surfaces abut and prevents the release button from falling out of the tubular body. When the release button is pushed to activate the power pack, the first and third contact surfaces abut and prevents the release button from returning back to its original position. A corresponding release button, a tubular body and a medicament delivery device are also provided.

### CROSS-REFERENCE TO RELATED APPLICATIONS

The present application is a U.S. National Phase application pursuant to 35 U.S.C. § 371 of International Application No. PCT/EP2022/079401 filed Oct. 21, 2022, which claims priority to European Patent Application No. 20206214.5 filed Nov. 3, 2021. The entire disclosure contents of these applications are herewith incorporated by reference into the present application.

### TECHNICAL FIELD

The present disclosure relates to the field of medicament delivery devices. In particular, the present disclosure relates to a power pack for such a device having a permanent locking of a release button.

### BACKGROUND

Autoinjectors and other types of medicament delivery devices are designed to allow for a user (e.g. a patient) to perform self-administration of a dose of a particular medicament in a controlled way. After positioning the device at the site of the body where the injection is desired, the user activates the device by pushing a release button. Pushing the release button releases a pre-compressed power spring which, when released, in turn drives a plunger rod towards the proximal end of the autoinjector. The plunger rod comes into contact with a syringe in which the medicament to be delivered is stored, and the movement of the plunger rod forces the medicament out of the syringe and into the injection site. In some autoinjectors, the movement of the plunger rod is also responsible for first inserting the needle into the bodily tissue at the injection site.

However, once the release button has been pushed in order to active the device, the release button is returned to its original position. This behavior can cause an uncertainty for the user whether the device has already been activated or not.

### SUMMARY

To at least partially solve the above identified problem with uncertainty created by the release button returning back to its original position after activation of the device, the present disclosure provides an improved power pack, an improved release button, an improved body for the power pack, and an improved medicament delivery device, as defined in the accompanying independent claims. Various alternative embodiments are defined in the dependent claims.

According to a first aspect of the present disclosure, a power pack for a medicament delivery device is provided. The power pack includes a tubular body extending along a longitudinal axis from a proximal end to a distal end. The tubular body includes a first protrusion which extends radially inwards from an inside wall of the tubular body and includes a first contact surface facing towards the proximal end. The power pack further includes a release button at least partially arranged within the tubular body and movable in relation to the tubular body along the longitudinal axis. The release button includes a flexible arm extending towards the proximal end. An end of the flexible arm includes a second protrusion extending radially outwards from the flexible arm and circumferentially aligning with the first protrusion. The second protrusion includes a second contact surface facing towards the distal end. In an envisaged first case, the release button further includes a third protrusion closer to the distal end along the longitudinal axis than the second protrusion and extending radially outwards from the flexible arm, circumferentially aligning with the second protrusion and including a third contact surface facing towards the distal end. In an envisaged second, alternative or complementary case, the tubular body further includes a third protrusion closer to the proximal end along the longitudinal axis than the first protrusion and extending radially inwards from the inside wall of the body, circumferentially aligning with the first protrusion and including a third contact surface facing towards the proximal end. In both cases, the flexibility of the flexible arm allows the release button to be pushed in the proximal direction along the longitudinal axis to at least a first position and a second position closer to the proximal end along the longitudinal axis than the first position, such that with the release button at the first position, a removal of the release button from the tubular body by pulling or pushing the release button towards the distal end is blocked by the first contact surface abutting the second contact surface, and such that with the release button at the second position and with the third protrusion included as part of the flexible arm (as in the envisaged first case), a pulling or pushing of the release button along the longitudinal axis towards the distal end and towards the first position is blocked by the first contact surface abutting the third contact surface or, with the release button at the second position and with the third protrusion included as part of the tubular body (as in the envisaged second case), a pulling or pushing of the release button along the longitudinal axis towards the distal end and towards the first position is blocked by the second contact surface abutting the third contact surface.

The present disclosure of the power pack according to the first aspect improves upon existing technology in that the use of multiple protrusions and contact surfaces on the flexible arm of the release button, or the use of multiple protrusions and contact surfaces on the tubular body, allows to not only keep the release button from falling out of the tubular body after manufacturing, but also to lock the release button in its pushed position after activation of the power pack and the medicament delivery device. As this prevents the release button from returning back to its original position after having been pushed, a user of the medicament delivery device can tell whether the medicament delivery device has already been used or not by looking at the position of the release button. This removes the uncertainty with commonly available other solutions discussed earlier herein. In addition, by modifying only the release button (i.e. by adding an extra protrusion to the flexible arm), the envisaged functionality of the present disclosure can be obtained without also having to modify the tubular body. This because, to prevent the release button from falling out of the body after insertion during manufacturing, the body already contains the single protrusion needed. Likewise, if instead adding the extra protrusion to the body of the power pack, only the body needs to be modified during manufacturing and no modifications also to the release button (including only a single protrusion on its flexible arm) are needed. Consequently, the envisaged solution of the present disclosure can be implemented with minimal changes to both design and manufacturing, resulting in both e.g. reduced costs and minimal added complexity.

In one or more embodiments of the power pack, the power pack may include a resilient component arranged to exert a force on the release button along the longitudinal axis towards the distal end. This may e.g. help to push the contact surfaces of the flexible arm and the tubular body together both in the first and second position of the release button, and thereby keep the release button from e.g. rattling around both before and after it has been used. The resilient component may also present a more likeable “pressing feel” to the user when operating the release button.

In one or more embodiments of the power pack, the resilient component may be a spring. The stiffness of the spring may be adapted in accordance with e.g. a desired required “pressing force” which the user has to apply in order to activate the device, and may also be adapted to e.g. a desired length of travel of the release button before activating the power pack and the medicament delivery device, etc.

In one or more embodiments of the power pack, the first contact surface, the second contact surface, and the third contact surface may all be perpendicular to the longitudinal axis. For example, the first, second and third contact surfaces may thus be parallel to each other when abutting, and all perpendicular to the direction of movement of the release button relative to the tubular body.

In one or more embodiments of the power pack, the release button may have a tubular body extending along the longitudinal axis. The flexible arm may be formed as part of an outer shell of the tubular body of the release button.

In one or more embodiments of the power pack, the tubular body of the power pack may include an inner tubular section arranged fixed relative to the tubular body and extending along the longitudinal axis. The power pack may further include a power spring and a plunger rod coaxially arranged at least partly within the inner tubular section. The power spring may be compressed and abut a proximal rim of the plunger rod such that, when the power spring is released by pushing the release button to the second position, the power spring drives the plunger rod along the longitudinal axis towards the proximal end. This configuration may provide one example mechanism for propelling/moving the plunger rod during activation of the power pack and the medicament delivery device. The power spring may for example be arranged on the outside of the plunger rod. In other embodiments, the power spring may be arranged on the inside of the plunger rod (which is then created hollow, with e.g. one or more internal rims for supporting at least a proximal end of the power spring).

In one or more embodiments of the power pack, the inner tubular section may include a distal flexible arm configured to abut a distal rim of the plunger rod to block the power spring from being released and the plunger rod from moving along the longitudinal axis towards the proximal end. The release button may include a protrusion extending from a distal end of the release button towards the proximal end of the tubular body. This protrusion may be configured to, as a result of pushing the release button to the second position, abut a distal surface of the flexible arm of the inner tubular section and cause a bending of this flexible arm radially outwards, and thereby prevent the blocking of the power spring from being released and the plunger rod from moving along the longitudinal axis towards the proximal end. This configuration may provide one example mechanism for activating the power pack and the medicament delivery device.

According to a second aspect of the present disclosure, a release button for a power pack for a medicament delivery device is provided. The release button is configured to be arranged movable within a tubular body of the power pack. The release button extends along a longitudinal axis from a proximal end to a distal end. The release button includes a flexible arm extending towards the proximal end, wherein an end of the flexible arm includes a second protrusion and a third protrusion both extending outwards from the longitudinal axis from the flexible arm. The second protrusion and the third protrusion includes a second contact surface and a third contact surface, respectively, both facing towards the distal end. The second protrusion is closer to the proximal end along the longitudinal axis than the third protrusion. Such a release button corresponds to the one in the first envisaged case of the power pack according to the first aspect.

In one or more embodiments of the release button, the release button may have a tubular body extending along the longitudinal axis. The flexible arm may be formed as part of an outer shell of the tubular body.

In one or more embodiments of the release button, the second contact surface and the third contact surface may both be perpendicular to the longitudinal axis. For example, the second and third contact surfaces may thus be parallel to each other, and both perpendicular to the direction of movement of the release button relative to the tubular body.

According to a third aspect of the present disclosure, a tubular body for a power pack for a medicament delivery device is provided. The tubular body is configured for arranging a release button of the power pack movably therein. The tubular body extends along a longitudinal axis from a proximal end to a distal end. The tubular body includes a first protrusion and a third protrusion both extending radially inwards from an inside wall of the body and circumferentially aligning. The first protrusion and the third protrusion includes a first contact surface and a third contact surface, respectively, both facing towards the proximal end. The third protrusion is closer to the proximal end along the longitudinal axis than the first protrusion. Such a tubular body corresponds to the one in the second envisaged case of the power pack according to the first aspect.

In one or more embodiments of the tubular body, the first contact surface and the third contact surface are both perpendicular to the longitudinal axis. For example. For example, the first and third contact surfaces may thus be parallel to each other, and both perpendicular to the direction of movement of the release button relative to the tubular body.

According to a fourth aspect of the present disclosure, a medicament delivery device is also provided. The medicament delivery device includes a power pack according to the first aspect.

Other objects and advantages of the present disclosure will be apparent from the following detailed description, the drawings and the claims. Within the scope of the present disclosure, it is envisaged that all features and advantages described with reference to e.g. power pack of the first aspect are relevant for, apply to, and may be used in combination with also any feature and advantage described with reference to the tubular body of the second aspect, the release button of the third aspect, and the medicament delivery device of the fourth aspect, and vice versa.

### BRIEF DESCRIPTION OF THE DRAWINGS

Exemplifying embodiments will now be described below with reference to the accompanying drawings, in which:

FIG. 1A schematically illustrates a perspective view of an embodiment of a medicament delivery device according to the present disclosure;

FIGS. 1B-1E schematically illustrate cross-sections of various embodiments of a power pack, a tubular body, and a release button according to the present disclosure, and

FIG. 1F schematically illustrates a perspective view of an embodiment of a release button according to the present disclosure.

In the drawings, like reference numerals will be used for like elements unless stated otherwise. Unless explicitly stated to the contrary, the drawings show only such elements that are necessary to illustrate the example embodiments, while other elements, in the interest of clarity, may be omitted or merely suggested. As illustrated in the Figures, the (absolute or relative) sizes of elements and regions may be exaggerated or understated vis-à-vis their true values for illustrative purposes and, thus, are provided to illustrate the general structures of the embodiments.

### DETAILED DESCRIPTION

Exemplifying embodiments of a medicament delivery device, a power pack, a tubular body, and a release button according to the present disclosure will now be described more fully hereinafter with reference to the accompanying drawings. The drawings show currently preferred embodiments, but the invention may, however, be embodied in many different forms and should not be construed as limited to the embodiments set forth herein; rather, these embodiments are provided for thoroughness and completeness, and fully convey the scope of the present disclosure to the skilled person.

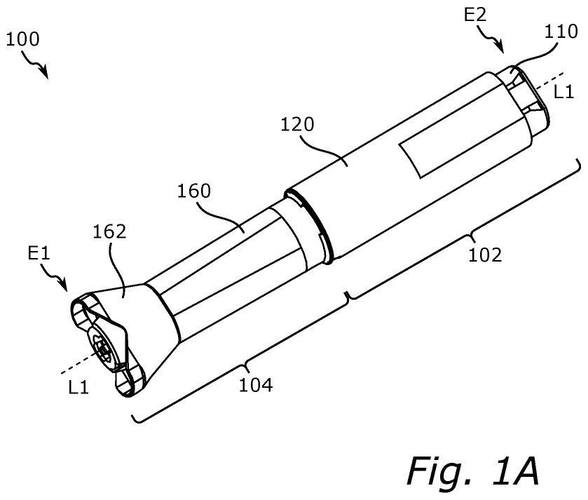

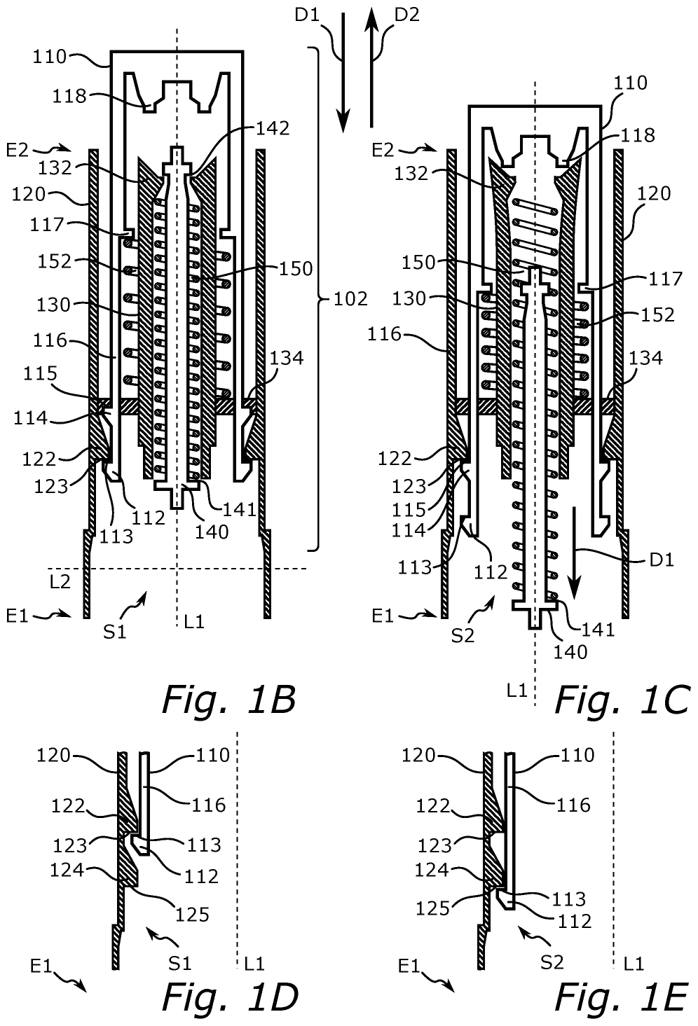

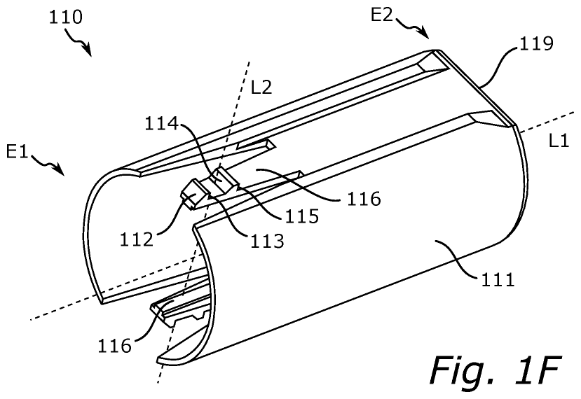

FIG. 1A schematically illustrates a medicament delivery device 100 according to one embodiment of the present disclosure, wherein the device 100 is yet to be fully assembled. Hereinafter, the terms “medicament delivery device” and “device” will be used interchangeably.

The device 100 can for example be manufactured as two sub-assemblies, namely a so-called power pack 102 and a so-called front assembly 104, which are joined together during final assembly of the device 100. In other embodiments, it is envisaged that the device 100 may arrive fully assembled from the factory, with the power pack 102 and the front assembly 104 already joined together. The power pack 102 and the front assembly 104 are arranged such that they both extend along a longitudinal axis L1, from a proximal end E1 to a distal end E2. The power pack 102 includes a tubular body 120 and a release button 110, both extending along the longitudinal axis L1. The release button 110 is at least partially arranged within the tubular body 120 and is movable along the longitudinal axis L1. Here, “movable” implies that the release button can, if not blocked by any other component of the power pack, be pushed at least in a proximal direction D1 relative to the tubular body 120. Such pushing of the release button 110 can be achieved e.g. by a user using a finger, such as a thumb, to apply a force on the release button in the proximal direction.

Although not explicitly indicated in FIG. 1A, it is to be understood that each one of the power pack 102 and the front assembly 104 can also be considered as having its own proximal and distal ends, and that each one of the power pack 102 and the front assembly 104 can also be considered as extending along its own longitudinal axis. When arranged as in FIG. 1A, the longitudinal axis of each sub-assembly aligns with the longitudinal axis L1 of the device as a whole. Likewise, the distal end of the power pack is also the distal end E2 of the device 100, while the proximal end of the front assembly 104 is the proximal end E1 of the device 100. The proximal end of the power pack 102 can be understood as the end of the power pack 102 being closest to the front assembly 104 and, similarly, the distal end of the front assembly 104 can be understood as the end of the front assembly 104 being closest to the release button 110. Similarly, each particular component of the power pack 102 and the front assembly 104 can be defined as having its own proximal and distal ends, and also its own longitudinal axis. When so doing, it can be assumed that the components are all arranged as indicated in FIG. 1A. The references L1, E1 and E2 will be used throughout the present disclosure to indicate such longitudinal axis, proximal end distal ends of each component, as needed. Even if the device 100 is fully assembled, it is envisaged that the various proximal and distal ends of the various components are still as they were before the device 100 was fully assembled, as illustrated in FIG. 1A. The device 100 can be fully assembled by e.g. pushing the distal end of the front assembly 104 into the proximal end of the power pack 102.

The front assembly 104 can optionally include a protective cap 162, which functions e.g. to protect a needle (not shown) of the front assembly 104 from being accessible by a user of the device 100. When the device 100 is to be used, the protective cap 164 can first be removed by the user. In some embodiments, the device 100 can be configured (using e.g. a rotator, also not shown) such that even with the protective cap 162 removed, the device 100 must first be pressed against the injection site before the release button 110 can be used to activate the power pack and the device, and cause the injection of the medicament into the body of the user.

The various components of the device 100 as envisaged by the present disclosure will now be described in more detail with reference to FIGS. 1B-1F.

FIG. 1B schematically illustrates a cross-section of an embodiment of the power pack 102 and parts of the power pack where the power pack 102 is not yet assembled with the front assembly. The power pack 102 includes the release button 110 and the tubular body 120. The tubular body 120 extends along the longitudinal axis L1 from a proximal end E1 to a distal end E2. The tubular body 120 has a protrusion 122 which extends radially inwards from an inside wall of the tubular body 120. Here, “radially inwards” is defined as pointing in a radial/lateral direction towards the longitudinal axis L1, while “radially outwards” is defined as pointing in a radial/lateral direction away from the longitudinal axis L1. It is envisaged that the tubular body 120 may not necessarily be cylindrical, and that a “radial” direction is then a “lateral” direction instead. The protrusion 122 of the tubular body 120 has a first contact surface 123 which faces towards the proximal end E1 of the tubular body 120.

The release button 110 is at least partially arranged within the tubular body 120, and is movable (with respect to the tubular body 120) along the longitudinal direction L1 of the tubular body 120. The release button 110 has a flexible arm 116 which extends towards the proximal end E1 of the tubular body 120. An end of the flexible arm 116 includes a second protrusion 112 which extends radially outwards from the flexible arm 116 and circumferentially aligns with the first protrusion 122 of the tubular body 120. The second protrusion 112 includes a second contact surface 113 which faces towards the distal end E2 of the tubular body 120.

In FIG. 1B, due to the flexibility of the flexible arm 116, the release button 110 has been pushed in the proximal direction D1 along the longitudinal axis L1 such that it is in a first position S1. In this first position S1, a removal of the release button 110 from the tubular body 120, by pulling or pushing the release button 110 towards the distal end E2, is blocked by the first contact surface 123 abutting the second contact surface 113, as illustrated in FIG. 1B.

In the particular embodiment illustrated in FIG. 1B, a resilient component (here in form of a spring) 152 is arranged to exert a force on the release button 110 along the longitudinal direction L1 towards the distal end E1, i.e. in the distal direction D2. This is achieved by a proximal end of the spring 152 abutting a structure 134 of the tubular body 120 which is fixed relative to the tubular body 120, and a distal end of the spring 152 abutting a rim 117 of the release button 110.

The force from the spring 152 helps to keep the first and second contact surfaces 123 and 113 abutting, and also provides a suitable “pressing force” which must be overcome in order for the user to press/push the release button 110 further in the proximal direction D1. In other embodiments, it is envisaged that the spring (or resilient component) 152 is not present, and/or that other suitable components are provided and arranged to provide such a “pressing force”.

Part of a mechanism of locking the power pack 102, a locking which may be removed by “activation” of the power pack 102, will be described in what follows. The tubular body 120 includes an inner tubular section 130 which is arranged within the tubular body 120 in a fixed manner, such that it cannot move with respect to the tubular body 120. The tubular body 120 may include e.g. on or more arms or other structures extending from its inside wall to the inner tubular section 130, in order to fixate the inner tubular section 130. One example of such a structure is the structure 134 on which the spring 152 may abut. Even if the spring 152 is not present, it is envisaged that the structure 134 may still be present in order to fixate the inner tubular section 130. The power pack 102 includes a power spring 150 and a plunger rod 140, which are both concentrically arranged at least partially within the inner tubular section 130. In the particular embodiment shown in FIG. 1B, the power spring 150 is arranged on the outside of the plunger rod 140. A distal end of the power spring 150 abuts e.g. part of the inner tubular section 130, while a proximal end of the power spring 150 abuts a proximal rim 141 of the plunger rod 140. With the release button in the first position S1, the power spring is at least partially compressed and exerts a force on the plunger rod 140 in the proximal direction D1 by the proximal end of the power spring 150 pressing against the proximal rim 141 of the plunger rod 140. To keep the plunger rod 140 from being propelled/driven by the power spring 150 in the proximal direction D1, a distal end of the inner tubular section 130 further includes a (distal) flexible arm 132. As shown in FIG. 1B, part of the flexible arm 132 abuts a corresponding distal rim 142 of the plunger rod 140, which blocks the plunger rod 140 from moving in the distal direction D2. As will be described in more detail later with reference to FIG. 1C, the holding/blocking of the plunger rod 140 caused by the flexible arm 132 and the distal rim 142 can be released by pressing of the release button 110 in the proximal direction D1.

In all the figures herein illustrating cross-sections, it should be noted that although some features are shown in a same cross-sectional plane, that must not necessarily be the case. For example, in the power pack 102 illustrated in FIG. 1B, it may for example be envisaged that the features related to the activation/locking of the power pack 102, including e.g. the flexible arm 132 of the inner tubular section 130, are instead arranged such that they would only show up in another cross-sectional plane, e.g. a cross-sectional plane perpendicular or otherwise angled with respect to the cross-sectional plane shown in FIG. 1B. However, for illustrative purposes only, the components described are assumed to be arranged such that a single cross-section can illustrate all described features.

In the particular envisaged configuration illustrated in FIG. 1B, the release button 110 further includes a third protrusion 114 which is arranged closer to the distal end E2 along the longitudinal axis L1 than the second protrusion 112. The third protrusion 114 extends radially outwards from the flexible arm, and circumferentially aligns with the second protrusion 122. The third protrusion 114 includes a third contact surface 115 which faces towards the distal end E2 of the tubular body 120. With the release button in the position S1, the third protrusion 114 does not necessarily interact with e.g. the tubular body 120, but may instead be used to cause a permanent lock of the release button 110 once the release button 110 is further pushed, as will now be described in more detail with reference to FIG. 1C.

FIG. 1C schematically illustrates a cross-section of the same power pack 102 as in FIG. 1B, but in another state wherein the release button 110 has been pushed (against the force provided by the spring 152) in the proximal direction D1 to a second position S2.

With the release button 110 pushed to the second position S2, the release button 110 causes an activation of the power pack 102 as follows. At a distal end of an inside of the release button 110, there is provided a protrusion 118 which extends from the distal end of the release button 110 towards the proximal end E1 of the tubular body 120. When the release button 110 is pushed to the second position S2, as shown in FIG. 1C, the protrusion 118 abuts a distal surface of the flexible arm 132 of the inner tubular section 130, and causes a bending of the flexible arm 132 radially outwards. When the flexible arm 132 is bent radially outwards, it is separated from the distal rim 142 of the plunger rod 140 and can no longer prevent/block the plunger rod 140 from being propelled/driven in the proximal direction D1 by the proximal force applied on the proximal rim 141 of the plunger rod 140 by the at least partially compressed power spring 150. As the power spring 150 expands, the plunger rod 140 is accelerated and moved along the longitudinal axis L1 in the proximal direction D1. This completes the activation of the power pack. The plunger rod 140 may then, in turn, reach and interact with e.g. a syringe/ampulla located in the front assembly, and cause an expulsion of a medicament from the syringe/ampulla. The movement of the plunger rod 140 may also, in some embodiments, be responsible for first causing a movement of a needle of the front assembly in the proximal direction, such that the needle is automatically inserted into the delivery site of the user before the medicament is expelled from the syringe/ampulla.

It should be noted that the features related to the activation of the power pack 102 (e.g. the inner tubular section 130, the flexible arm 132, the plunger rod 140, the rims 141 and 142, the power spring 150, etc.) and their interactions are here discussed only for reasons of completeness, even though their exact function, arrangement and similar is not directly relevant to the core of the present disclosure, which is the provision of multiple protrusions and contact surfaces either on the flexible arm 116 of the release button 110 or, as will be described later herein with reference to FIGS. 1D and 1E, on the inside wall of the tubular body 120.

Simultaneously with activating the power pack 102, the movement of the release button 110 to the second position S2 causes the flexible arm 116 to move further in the proximal direction D1, and the flexibility of the flexible arm 116 allows the third protrusion 114 to move past the first protrusion 122. Once the third protrusion 114 has moved past the first protrusion 122, the flexible arm 116 may once again “snap back” into an unbent state, and the third contact surface 115 is then closer to the proximal end E1 than the first contact surface 123 is. This new arrangement of the first and third contact surfaces 123 and 115 now has the effect that a pulling or pushing of the release button 110 along the longitudinal axis L1 towards the distal end E2 and towards the first position S1 is blocked by the first contact surface 123 abutting the third contact surface 115. Consequently, once the release button 110 has been pushed to activate the power pack 102 and the device 100, the abutting of the first and third contact surfaces 123 and 115 locks the release button 110 in its pushed position, and keeps it from returning back to its original position. As described earlier herein, this removes the uncertainty for the user about whether the power pack 102 and the device 100 has already been activated or not.

It should also be noted that as the present disclosure, in the present embodiment of the power pack, envisages to modify only the flexible arm 116 of the release button 110 (by adding the third protrusion 114 in addition to the second protrusion 114 already used for other purposes, such as preventing the release button 110 from falling out of the tubular body 120), no other components of the power pack 102 and the device 100 needs to be modified to achieve the envisaged effect. Thus, the solution offered herein is easy to implement, and does not come with any substantial addition of complexity or substantial increase in production costs. Already produced devices may for example be upgraded by retrofitting therein the new, improved release button 110.

The envisaged effect of locking the release button 110 in its pushed position S2 can be obtained also by instead modifying the tubular body 120, as will now be described in more detail with reference to FIGS. 1D and 1E.

FIG. 1D schematically illustrates a cross-section of a part of an alternative embodiment of the power pack 102, showing only the proximal end of the flexible arm 116 of the release button 110 and the corresponding part of the tubular body 120 where the protrusion 122 is provided. In this embodiment, the flexible arm 116 of the release button 110 only has the second protrusion 112 with the second contact surface 113, while the tubular body 120 instead includes the third protrusion 124. The third protrusion 124 is closer to the proximal end E1 along the longitudinal axis L1 than the first protrusion 122 is, and extends radially inwards from the inside wall of the tubular body 120. The third protrusion 124 circumferentially aligns with the first protrusion 122, and includes a third contact surface 125 which faces towards the proximal end E1. In FIG. 1D, the release button is in the first position S1, and the abutting of the first contact surface 123 and the second contact surface 113 prevents the release button 110 from falling out of the tubular body 120, by e.g. being pushed or pulled in the distal direction. Other than moving the third protrusion from the flexible arm of the release button to the tubular body, the remaining components and functionality of e.g. the tubular body 120 and the release button 110 may be identical to those described above with reference to FIGS. 1B and 1C.

FIG. 1E schematically illustrates a cross-section of the same part of the alternative embodiment of the power pack, but with the release button 110 now pushed to the second position S2. Such a pushing is still possible due to the flexibility of the flexible arm 116, as that the second protrusion 112 can be moved past also the third protrusion 124 (when pressing on the release button 110 in the proximal direction along the longitudinal axis L1). In the second position S2, the second contact surface 113 is now closer to the distal end E2 of the tubular body than the third contact surface 125 is. This arrangement of the second and third contact surfaces 113 and 125 also has the effect that a pulling or pushing of the release button 110 along the longitudinal axis L1 towards the distal end and towards the first position S1 is blocked by the second contact surface 113 abutting the third contact surface 125. Consequently, also in this embodiment, once the release button 110 has been pushed to activate the power pack and the device, the abutting of the first and third contact surfaces 123 and 115 locks the release button 110 in its pushed position, and keeps it from returning back to its original position. As described earlier herein, this once again removes the uncertainty for the user about whether the power pack 102 and the device 100 has already been activated or not.

In some embodiments of the power pack 102 described herein e.g. with reference to FIGS. 1B-1E, it may be envisaged that the contact surfaces (such as the first contact surface 123, the second contact surface 113 and the third contact surface 115 or 125) are all perpendicular to the longitudinal axis L1 (i.e. to the direction of movement of the release button 110 within the tubular body 120).

An exemplary embodiment of a release button 110 according to the present disclosure will now be described in more detail with reference to FIG. 1F.

FIG. 1F schematically illustrates a release button 110. The release button 110 illustrated in FIG. 1F may for example correspond to or be equal to the release button 110 illustrated in any of FIGS. 1B and 1C. The release button 110 has a tubular body 111 which extends along a longitudinal axis L1 from a proximal end E1 to a distal end E2. The release button includes a flexible arm 116, which extends towards the proximal end E1. The flexible arm 116 is formed as part of an outer shell of the tubular body 111. The tubular body 111 may for example be cylindrical, oval, or have any other suitable shape. Preferably, the shape of the tubular body 111 is configured to match the shape of the inside of the tubular body 120 of the power pack itself, in which the release button 110 is to be arranged movably, taking into account any deviations in shape caused by features such as guiding ribs, grooves or similar which may be needed to properly guide and hold the release button 110 within the tubular body of the power pack. The outer shape of the tubular body 111 of the release button 110 need not also, for example, be completely circular, oval, etc., but may have similar deviations in shape caused by e.g. various guiding ribs, grooves, or similar, which may interact with the corresponding features on the inside of the tubular body of the power pack.

In the example shown in FIG. 1F, the release button 110 includes two flexible arms 116, where an end of each flexible arm includes a second protrusion 112 and a third protrusion 114 which both extends (radially/laterally) outwards away from the longitudinal axis from each flexible arm 116. The second protrusion 112 includes a second contact surface 113, and the third protrusion 114 includes a third contact surface 115. Both the second and third contact surfaces 113 and 115 face towards the distal end E2. The second protrusion 112 is arranged closer to the proximal end E1 along the longitudinal axis L1 than the third protrusion 114 is. In other embodiments, it is envisaged that the number of flexible arms may be different, and include e.g. only one flexible arm, or more than two flexible arms, and that the tubular body may be modified accordingly and have a number of protrusion matching the number of flexible arms 116 of the release button 110.

At the distal end E2 of the release button 110, there may for example be provided a pressing surface 119, against which the user may place e.g. a finger (such as a thumb) in order to push the release button 110 to activate the device. The release button 110 may also, in some embodiments, include various other structures, such as e.g. one or more protrusions (not shown) for interacting with a rotator of the front assembly of the power pack, or similar, to provide other functionality of the device than the envisaged permanent lock of the release button 110 once pushed.

As described earlier herein, that a particular surface “faces towards” or “faces away from” an axis (or a surface/wall) does not require that e.g. a surface normal of the particular surface is exactly perpendicular to the axis. Instead, as long as the surface normal, or e.g. an average surface normal, or similar, of the particular surface has at least one component which is perpendicular to, and points towards, the axis in question, the particular surface is considered as facing towards that axis. Phrased differently, as long as e.g. an infinitely long vector starting at the particular surface and oriented in a same direction as a surface normal of the particular surface at some point intersects the axis, that particular surface is said to be facing towards the axis. Similarly, any particular surface that does not fulfill the above requirements are said to be “facing away” from the axis. The same reasoning applies also when saying that a particular surface faces away form/towards a particular other object such as a surface, or wall (such as e.g. the inside wall of the tubular body of the power pack, etc.).

In summary of FIGS. 1A-1F, it is concluded that the various components of the device 100, and in particular of the power pack 102 (such as the release button 110 and the tubular body 120) as envisaged herein are configured to interact such that: i) when the release button 110 is assembled in the tubular body 120 but not yet pushed to activate the power pack 102 and the device 100, the interaction of a protrusion on the tubular body 120 and a protrusion on the flexible arm 116 of the release button 110 prevents the release button 110 from falling out of the tubular body 120, and ii) after the release button 110 has been pushed to activate the power pack 102 and the device 100, an additional protrusion (located either on the flexible arm 116 of the release button 110 or on the inside wall of the tubular body 120) interacts with an already existing protrusion (either on the release button 110 or on the flexible arm 116) to also provide a locking of the release button 110 in its pushed position, such that it does not return back to its original position, and such that the user can tell whether the power pack 102 and the device 100 has already been activated by looking at the (position of) release button 110. These features together provide an improved device, power pack, release button and tubular body, wherein the uncertainty for the user about the current state of the device is reduced or even removed.

Although not explicitly disclosed in any drawings, or at least not explicitly further described in the description so far, it is herein envisaged that the medicament delivery device may also include additional components, including e.g. the protective cap for the delivery members, an ampulla for storing the dose of medicament, one or more guiding rods and or other structures for the arrangement of a delivery member cover, a rotator for blocking pushing of the release button before e.g. the delivery member cover is pressed against the injection site, etc. It is envisaged that any such components are not directly relevant for describing the idea behind the present disclosure, and any further illustration and/or discussion of such additional components has therefore, for reasons of clarity, been left out of the present disclosure.

Medicament delivery devices as envisaged herein may for example be autoinjectors containing insulin, allergy medicaments, epinephrine, migraine medicaments, atropine, or any other medicament/drug for which the use of a medicament delivery device as described herein is suitable. The device can e.g. be of a single-use type (i.e. disposable). Delivery members may include needles, parts of syringes, syrettes, or other suitable structures.

In the present disclosure, the term “longitudinal axis” of the device refers to an axis extending from a proximal end of the device to a distal end of the device, typically a central axis along the device in the direction of longest extension of the device. The term “distal end” refers to the part/end of the device, or the parts/ends of the members thereof, which under use of the device is/are located furthest away from the dose delivery/injection site. Correspondingly, the term “proximal end” refers to the part/end of the device, or the parts/ends of the members thereof, which under use of the device is/are located closest to the dose delivery/injection site. As described further above, this also apply to the various components of the device itself, such as the power pack, the front assembly, the release button, the tubular body of the power pack, etc., which can all be considered as each having their own longitudinal axis (where applicable), and which, when assembled in the device, can all be considered as having also their own corresponding proximal and distal ends.

Although features and elements may be described above in particular combinations, each feature or element may be used alone without the other features and elements or in various combinations with or without other features and elements. Additionally, variations to the disclosed embodiments may be understood and effected by the skilled person in practicing the claimed invention, from a study of the drawings, the disclosure, and the appended claims.

In the claims, the words “comprising” and “including” does not exclude other elements, and the indefinite article “a” or “an” does not exclude a plurality. The mere fact that certain features are recited in mutually different dependent claims does not indicate that a combination of these features cannot be used to advantage.

1-13. (canceled)

14. A power pack for a medicament delivery device, comprising:
a tubular body extending along a longitudinal axis from a proximal end to a distal end, wherein the tubular body includes a first protrusion which extends radially inwards from an inside wall of the tubular body and includes a first contact surface facing towards the proximal end; and
a release button at least partially arranged within the tubular body and movable in relation to the tubular body along the longitudinal axis, wherein the release button includes a flexible arm extending towards the proximal end, wherein an end of the flexible arm includes a second protrusion extending radially outwards from the flexible arm and circumferentially aligning with the first protrusion, wherein the second protrusion includes a second contact surface facing towards the distal end,
wherein
the release button further includes a third protrusion closer to the distal end along the longitudinal axis than the second protrusion and extending radially outwards from the flexible arm, circumferentially aligning with the second protrusion and including a third contact surface facing towards the distal end, or
the tubular body further includes a third protrusion closer to the proximal end along the longitudinal axis than the first protrusion and extending radially inwards from the inside wall of the body, circumferentially aligning with the first protrusion and including a third contact surface facing towards the proximal end, and
wherein the flexibility of the flexible arm allows the release button to be pushed in the proximal direction along the longitudinal axis to at least a first position and a second position closer to the proximal end along the longitudinal axis than the first position, such that with the release button at the first position, a removal of the release button from the tubular body by pulling or pushing the release button towards the distal end is blocked by the first contact surface abutting the second contact surface, and such that with the release button at the second position and with the third protrusion included as part of the flexible arm, a pulling or pushing of the release button along the longitudinal axis towards the distal end and towards the first position is blocked by the first contact surface abutting the third contact surface or, with the release button at the second position and with the third protrusion included as part of the tubular body, a pulling or pushing of the release button along the longitudinal axis towards the distal end and towards the first position is blocked by the second contact surface abutting the third contact surface.

15. The power pack according to claim 14, further comprising a resilient component arranged to exert a force on the release button along the longitudinal axis towards the distal end.

16. The power pack according to claim 15, wherein the resilient component is a spring.

17. The power pack according to claim 14, wherein the first contact surface, the second contact surface, and the third contact surface are all perpendicular to the longitudinal axis.

18. The power pack according to claim 14, wherein:
the release button has a tubular body extending along the longitudinal axis, and
the flexible arm is formed as part of an outer shell of the tubular body of the release button.

19. The power pack according to claim 14, wherein:
the tubular body of the power pack includes an inner tubular section arranged fixed relative to the tubular body and extending along the longitudinal axis, and
wherein the power pack further comprises:
a power spring and a plunger rod coaxially arranged at least partly within the inner tubular section, wherein the power spring is compressed and abuts a proximal rim of the plunger rod such that, when the power spring is released by pushing the release button to the second position, the power spring drives the plunger rod along the longitudinal axis towards the proximal end.

20. The power pack according to claim 19, wherein:
the inner tubular section includes a distal flexible arm configured to abut a distal rim of the plunger rod to block the power spring from being released and the plunger rod from moving along the longitudinal axis towards the proximal end, and
the release button includes a protrusion extending from a distal end of the release button towards the proximal end of the tubular body, and configured to, as a result of pushing the release button to the second position, abut a distal surface of the flexible arm of the inner tubular section and cause a bending of the flexible arm radially outwards, and thereby prevent the blocking of the power spring from being released and the plunger rod from moving along the longitudinal axis towards the proximal end.

21. A release button for a power pack for a medicament delivery device, the release button being configured to be arranged movable within a tubular body of the power pack, extending along a longitudinal axis from a proximal end to a distal end, and comprising:
a flexible arm extending towards the proximal end, wherein an end of the flexible arm includes a second protrusion and a third protrusion both extending outwards from the longitudinal axis from the flexible arm,
wherein the second protrusion and the third protrusion includes a second contact surface and a third contact surface, respectively, both facing towards the distal end,
wherein the second protrusion is closer to the proximal end along the longitudinal axis than the third protrusion.

22. The release button according to claim 21, wherein:
the release button has a tubular body extending along the longitudinal axis, and
the flexible arm is formed as part of an outer shell of the tubular body.

23. The release button according to claim 21, wherein the second contact surface and the third contact surface are both perpendicular to the longitudinal axis.

24. A tubular body for a power pack for a medicament delivery device, the tubular body being configured for arranging a release button of the power pack movably therein, extending along a longitudinal axis from a proximal end to a distal end, and comprising:
a first protrusion and a third protrusion both extending radially inwards from an inside wall of the body and circumferentially aligning,
wherein the first protrusion and the third protrusion includes a first contact surface and a third contact surface, respectively, both facing towards the proximal end,
wherein the third protrusion is closer to the proximal end along the longitudinal axis than the first protrusion.

12. The tubular body according to claim 24, wherein the first contact surface and the third contact surface are both perpendicular to the longitudinal axis.

13. A medicament delivery device, comprising a power pack according to claim 14.
